(19) United States
(12) Patent Application Publication
(10) Pub. No.: US 2008/0269870 A1
(43) Pub. Date: Oct. 30, 2008

(54) Method for Preparing Medical Stents

(75) Inventors: Jari Ruuttu, Billnas (FI); Olli Saarniaho, Vaasa (FI); Harry Asonen, Tampere (FI); Jarno Kangastupa, Kangasala (FI); Kalle Yla-Jarkko, Hameenlinna (FI); Arto Salokatve, Tampere (FI)

Correspondence Address: YOUNG & THOMPSON, 209 Madison Street, Suite 500, ALEXANDRIA, VA, 22314, US

(73) Assignee: VESLATEC OY, VAASA (FI)

(21) Appl. No.: 11/791,654

(22) PCT Filed: Nov. 22, 2005
(86) PCT No.: PCT/FI2005/000494

§ 371 Date: Apr. 11, 2008

(30) Foreign Application Priority Data
Nov. 25, 2004 (FI) 20041515

(51) Int. Cl. A61F 2/82; B29C 35/08

(52) U.S. Cl. 623/1.15; 264/400

(57) ABSTRACT

A method for preparing stents, with a stent blank subjected to a work process, in which the desired pattern is cut through the stent blank by evaporating the stent material with a diode-pumped fibre laser. The used fibre laser is preferably a picosecond laser having a minimum power of 20 W and a repetition frequency above 1 MHz.

### FIELD OF THE INVENTION

This invention relates to a method for preparing medical stents by diode-pumped fibre laser technology. The method of the invention allows preparation of stents from metals, plastics and biopolymers, among other materials.

### STATE OF THE ART

### Stents

A “stent” is a device inserted at a blockage in a blood vessel or any other tubular structure for keeping the passageway open. Stents are common in medical use in the treatment of various vascular or duct blockages. A stent is a small tubular mesh, which is installed in a stretched state within a blood vessel to be attended to e.g. after balloon angioplasty or mechanical plaque removal. The stent has the function of keeping the treated vascular location open.

Stents can be classified according to the used materials, the structure, the installation manner and the surgical theme of application, and also according to temporary or permanent purposes of use.

A coronary stent (referred to as stent here) is a stent which installed in the coronary artery and which can be self-expanding, balloon dilatable or a thermal memory stent. The material used in a stent may consist of stainless steel, nitinol, polymer-coated stainless steel, medicine-coated stainless steel, biopolymers, coated polymers, memory metals or any other coated or uncoated material.

Stents usually have a wall thickness in the approximate range from 0.1 mm to 0.15 mm and their length is typically in the range from 15 mm to 30 mm. Depending on the object of use, stents have a typical diameter in the approximate range from 0.8 to 2 mm or more, depending on the object of use.

U.S. Patent Application 2004/0024485 A1 discloses the use of laser in stent manufacture, with the stent made in pressurised oxygen. This is said to increase the burning effect of the laser beam. The use of water is further set forth for cooling purposes.

U.S. Pat. No. 6,696,667 B1 sets forth that thermal damages caused by a laser beam can be avoided by shifting the laser beam focus on the x axis (longitudinally) of the stent blank using a planar scanner so rapidly that no material plasma has time or is allowed to form. The reference also describes known laser applications, such as Nd:YAG, EXCIMER, copper steam laser, lamp or diode pumped lasers and phemtosecond laser.

Phemtosecond laser typically has a pulse length of 150-300 phemtoseconds. This naturally does not cause serious thermal damage, however, phemtosecond lasers have the drawback of a slow machining process.

U.S. Pat. No. 6,696,667 B1 discloses a total pulse length of 100 μs (microseconds) in the use of Q-Switch-Nd:YAG:laser. This apparatus is at present the most frequently used laser apparatus in stent manufacture.

The same reference hence sets forth that stent cutting is specifically based on burning and that no plasma is produced because it damages the stent.

U.S. Pat. No. 6,369,355 discloses a method for manufacturing stents based on pattern formation in the stent material by means of laser. This pattern formation is explicitly based on burning, i.e. melting. The reference states that the laser beam focus can be reduced from 1.06 μ (microns) to about the half, more specifically to 0.532 microns (μ). The laser used is an Nd:YAG laser equipped with a Q switch and having a laser pulse length under 100 ns (nanoseconds). The pulse repetition frequency is indicated as up to 40 KHz.

In accordance with this reference, reduction of the laser beam focus allegedly reduces deformation of the metal part of the stent. According to the reference, carbon dioxide (CO₂) or oxygen is sprayed towards the laser beam machining location through a separate nozzle connected to the laser apparatus.

Laser cutting of stent blanks based on burning, i.e. melting, always involves thermal transfer to the remaining parts of the stent material. It cannot be acted on with any cooling method, since this would hamper the actual operation, i.e. stent cutting.

In addition, it is widely known to use a combination of a rotary movement and a longitudinally reciprocating movement of the stent during laser cutting (CNC).

Further, all current stent manufacture processes require the following work steps: a) ultrasonic washing of the stent, b) dissolving in TKL for more than 8 minutes, c) electrochemical polishing, d) repeated ultrasonic washing, e) sterilisation by the process and f) metal tempering, preheating by means of a temperature in the approximate range +900-1000° C.

### SUMMARY OF THE INVENTION

The purpose of use of stents requires an extremely high-precision manufacturing process. Consequently, current methods are extremely costly, complicated and slow. Despite consistent developments in the manufacturing processes, current stents still do not have good quality.

The first problem relating to manufacturing processes is caused by the work process used for cutting a metal stent. The use of laser is the most popular way of cutting a stent, since no other thermal method is applicable due to the small size of the piece. This is why the laser type is a vital issue in the choice of manufacturing method.

A typical stent pattern is illustrated in FIGS. 7a and b, whereas FIG. 8 shows a prior art cutting device and work process. The first problem arises during the cutting of such patterns. In the use of conventional laser techniques, a major portion of the energy consumed in the process is conducted to the machined piece in the form of thermal heat, causing various deformations of the stent material, which subsequently affect the properties of the stent and the materials used in it.

Such effects are fragility of the treated material and a broken atomic structure. To avoid such effects, the finished stent has been subjected to preheating at a temperature of +950° C. The preheating process has a duration of about four hours.

If the metal tube is intact, preheating normally has a beneficial effect. However, after laser cutting through the wall of the stent blank, the wall only has a thickness of about 0.1 mm.

The laser beam used in prior art laser apparatuses has a long pulse and high energy, in other words, the practical work process is performed by burning, i.e. melting the metal at the location where the laser beam penetrates through the material.

Then the temperature is 2000-6000° C. in the area influenced by the laser beam, and a major portion of the laser beam energy is transferred into the stent material proper. Such a thermal chock has a very detrimental effect on the quality of the basic stent blank material.

The most frequently used laser type is an Nd:YAG laser. The pulse length is then of the order of microseconds and the repetition frequency is in the range 50 to 2000 Hz. An Nd:YAG laser is a crystal laser which may be lamp or diode pumped. The laser pulse is hence long, generating consequently a thermal shock in the metal, resulting in poorer stent metal quality.

When this technique is used, very sharp edges are formed at the cutting location, and due to the molten metal the cutting trace will be indefinite. In addition, loose parts remain adhered to the stent during cutting, and such parts should necessarily be removed before the stent is delivered to its purpose of use.

Consequently, there have been efforts to resolve the problems above by subjecting the stent to an electro-catalytic polishing process. However, the result of this work step is not reliable.

A third problem resides in the fact that the starting material, the stent blank tube, has become hard in the course of the manufacturing process, and when a hard metal is subjected to a local thermal shock, the metal quality is substantially impaired and considerable stresses are generated.

After the work processes above, the laser-machined and electro-catalytically polished stent is placed in a tempering furnace, where the temperature is raised to about +950° C. The preheating process has an approximate duration of 4 hours. There will still remain considerable stresses in the stent, and it has not burnt into a straight shape, having also an irregular surface structure.

FIGS. 6 and 8 illustrate a prior art stent manufacturing process and cutting apparatus.

Known methods are illustrated in FIGS. 6 and 8. FIGS. 7a and 7b illustrate typical stents.

The invention that has now been found resolves the problems mentioned above.

The invention relates to a method for manufacturing stents, in which stent materials are laser machined so that the stent blank is subjected to a work process, where the desired pattern is cut through the stent blank by evaporating the stent material with a diode-pumped fibre laser.

The invention that has now been found is based on the surprising observation that diode-pumped pulse lasers, especially high-effect lasers of at least 20 W picoseconds, are applicable to high-quality stent manufacture. The manufacture is appreciably faster than in previous methods, allowing several of the work steps required in previous methods to be totally omitted. In this manner, stent manufacture is considerably more affordable than before.

Since a stent can now be manufactured in vertical position, unlike previous methods, it will not be subject to the same forces caused by gravitation and tending to bend the stent as in prior art methods.

The invention also offers the possibility to integrate the sterilisation, quality control and packaging steps in a closed production process. This allows for a high-quality and reliable overall process well adapted to the purpose of use of stents.

### FIGURES

FIG. 1. A stent manufacturing apparatus in accordance with the invention, in which the work space is a sealed vacuum chamber (1) made of metal, for instance.

FIG. 2. A station (15) for machining a stent blank (19).

FIG. 3. Illustration of the operation of a setting unit (7).

FIG. 4. Top view of the operation of the setting unit (7).

FIG. 5. Graphic scheme of a stent manufacturing process.

FIG. 6. Graphic scheme of a prior art stent manufacturing process.

FIG. 7a. A typical stent pattern.

FIG. 7b. A typical stent pattern.

FIG. 8. A prior art stent cutting apparatus and machining process.

### DETAILED DESCRIPTION OF THE INVENTION

This invention relates to a method for manufacturing stents, in which stent materials are laser machined so that the stent blank is subjected to a work process, in which the desired pattern is cut through the stent blank by evaporating the stent material with a diode-pumped fibre laser.

In one embodiment of the invention, the power of the diode-pumped fibre laser is at least 20 W, preferably at least 50 W and most advantageously at least 100 W. Such a diode-pumped laser is a picosecond or phemtosecond laser, preferably a picosecond laser.

A picosecond laser is preferably a modularly reinforced and distributed fibre-reinforced picosecond laser. Such a diode-pumped picosecond fibre laser further uses a pulse frequency above 1 MHz, preferably above 10 MHz and most advantageously above 40 MHz.

Using a modularly reinforced distributed pulse laser method, one can achieve e.g. a net laser power of 1000 W, which can be distributed over e.g. ten stent manufacturing modules without increasing the price of this laser apparatus. The laser beam can be conducted to the work location over a fibre and via an optically corrected scanner.

In a particularly advantageous embodiment of the invention, the stent is cut with a 100 W picosecond laser, the pulse length being approximately 20-30 ps, the repetition frequency approximately 20 MHz and the individual pulse power about 5 μJ.

Depending on the material, the pulse power is approximately 1-15 J/cm² in the method of the invention.

In one embodiment of the invention, the stent blank is made of metal or metal compound. In this case, the stent blank is preferably preheated to a soft state before the pattern is cut through the stent blank with a diode-pumped fibre laser.

In a second preferred embodiment of the invention, the stent blank is made of polymer, biopolymer or a ceramic material. The stent blank can be made of other materials as well. The stent blank does not necessarily consist of one single material. The stent blanks made of the materials mentioned above can also be coated with a metal, a metal compound, a polymer (plastic) or say, a biopolymer. In addition, the finished stent can be coated with a pharmaceutical product.

In the method of the invention, a laser beam is preferably directed to a work piece, i.e. a stent blank, by means of an optically corrected planar scanner. The actual work process is preferably performed on a vertically positioned work piece.

In a preferred embodiment of the invention, an automated stent blank reserve is used. All the work processes in the stent manufacture are preferably automated and they include all the necessary work processes, including packaging.

The method of the invention preferably uses a sealed vacuum chamber as the work space, where the process may take place under gas atmosphere, vacuum, pressurisation, or a combination or joint use of these.

A particularly advantageous result is achieved if the entire work process is performed in vacuum and/or under gas atmosphere, because this allows the entire process to be carried out continuously or in separate work steps, as necessary.

In a preferred embodiment of the invention, the stent blank is completely machined over its entire length, and is only then cut to its final length.

In the method of the invention, the stent blanks are preferably transferred to the work chamber in a hot and sterile state. One advantageous manner of transferring the stent blanks to the work chamber is transferring them in a special cassette. This allows up to several hundreds of stent blanks to be loaded at once into the apparatus.

In a further preferred embodiment of the invention, stent quality control, packaging and code affixing are performed automatically in a closed and sterile space. Such a space may contain vacuum, gas, UV light and heat, or combinations of these.

The method of the invention should not be restricted to stents alone, since it is applicable to the cutting of other medical implants, such as screws made of biopolymer or metal.

Consequently, the method of the invention differs from known methods by the very fact that the starting material is a preheated stent blank of full length and having the final softness, from which the actual stents are formed. The stent blank may be previously polished, if considered necessary, both on the inside and the outside, using e.g. an electro-catalytic process. Polishing tubes having a length of 200-300 mm is considerably easier and more economical than polishing discrete machined stents having a length of 15-25 mm.

It is essential that, in accordance with the new method, stent blanks can be machined when in a preheated state, i.e. soft, without causing problems like those relating to the prior art process: thermal shock, modification of the stent material, flashes, a roughened surface, etc.

This is consequently possible on the following conditions: the fibre-reinforced laser apparatus is a) diode pumped, b) has high frequency 1-100 MHz, c) is a picosecond laser, whose d) pulse power is adequate, such as 1-15 J/cm² (joule/square cm). All the energy directed to the stent blank will be consumed merely by evaporation of material to be removed in the cutting groove. In this situation, the remaining stent material will not be subject to any kind of thermal effect, nor will the properties of the preheated soft metal change, and the cut trace will be neat without flashes or any other effects on the surface. Since, unlike previous methods, the stent can now be manufactured in vertical position, it will not be subject to forces caused by gravitation and tending to bend the stent like those occurring in prior art methods.

Microlinears, i.e. robotics, carries out all the work processes quite automatically on the basis of a provided file, and on top of this, packaging and sterilisation have now been integrated in the automatic manufacturing process. In addition, the finished stent can now be packaged under gas atmosphere, with the protective gas remaining in the stent package after sealing, thus naturally ensuring complete sterility over a very long period. The package can further be equipped with an indicator indicating function of the protective gas, in other words, that the stent is uninfected by bacteria or virus.

The invention that has now been found consequently allows for the manufacture of high-quality stents at significantly lower cost than before, while excluding such stent treatment steps that were previously required in the manufacturing process. The stent manufacturing speed will accelerate dramatically and the potential integration of sterilisation, quality control and packaging steps in the closed production process allows for a high-quality and safe overall process, which is well adapted to the purpose of use of the stents.

### EXAMPLES

The method of the invention for manufacturing stents is described below, yet without restricting the invention to the examples given here.

### Example 1

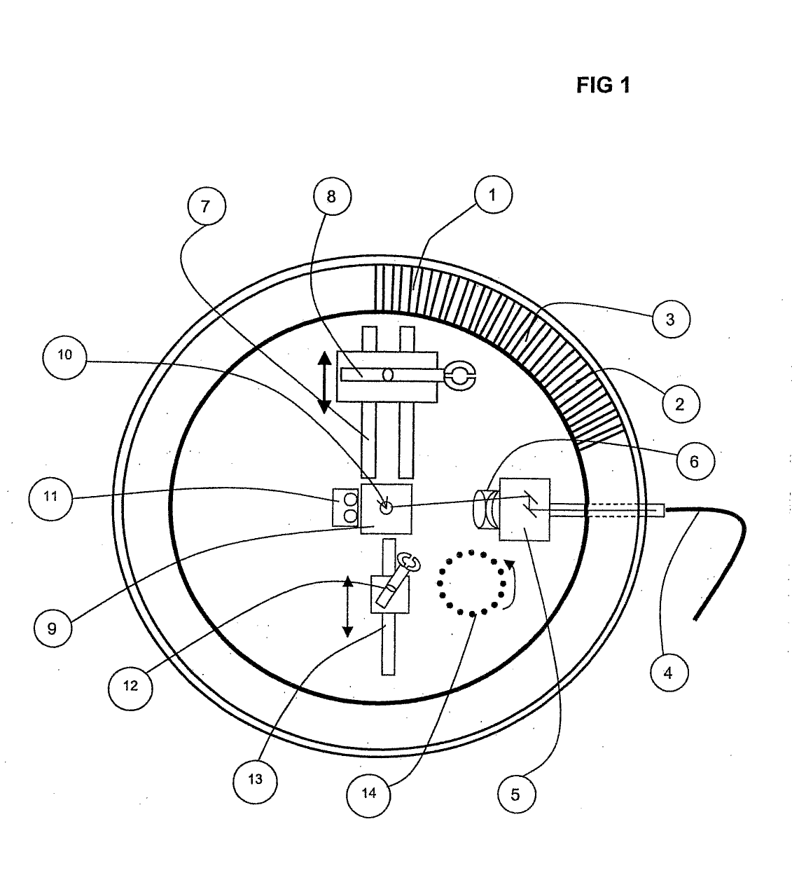

This example describes a method for manufacturing stents in accordance with the invention, in which the work space is a sealed vacuum chamber (1), which is made of metal, for instance, and which may have any shape, FIG. 1. If a stent blank cassette (2) has been placed in the chamber, a round cassette and a round recipient will result in a more advantageous design.

The stent cassette (2) may move freely in the peripheral direction of the chamber (1). It is preferably provided with a linear or step motor. This allows control of the movement of the cassette (2) such that the stent blanks (3) within the cassette (2) are fitted in the correct position, from where the transfer and setting unit (7) can retrieve (8) them.

When the unit for transferring and setting stent blanks (7) has gripped a stent blank (3), it engages it into a hole (10) of the actual stent machining station (8) in “vertical position”. The stent setting unit (11) attends to setting the stent blank to the correct height in the stent machining station (9), so that the laser beam (4) passes through the optically corrected scanner (6) to the stent blank, which engages the machining station, (9). When the stent is finished, the microrobot/manipulator (12) grips the finished stent and shifts it to an intermediate storage (14), which can also be an automatic packaging station if the chamber (1) is equipped with protective gas. The automatic packaging station may comprise also quality control, whose function is performed by a fully automated unit equipped with a digital camera and capable of distinguishing even minuscule flaws.

### Example 2

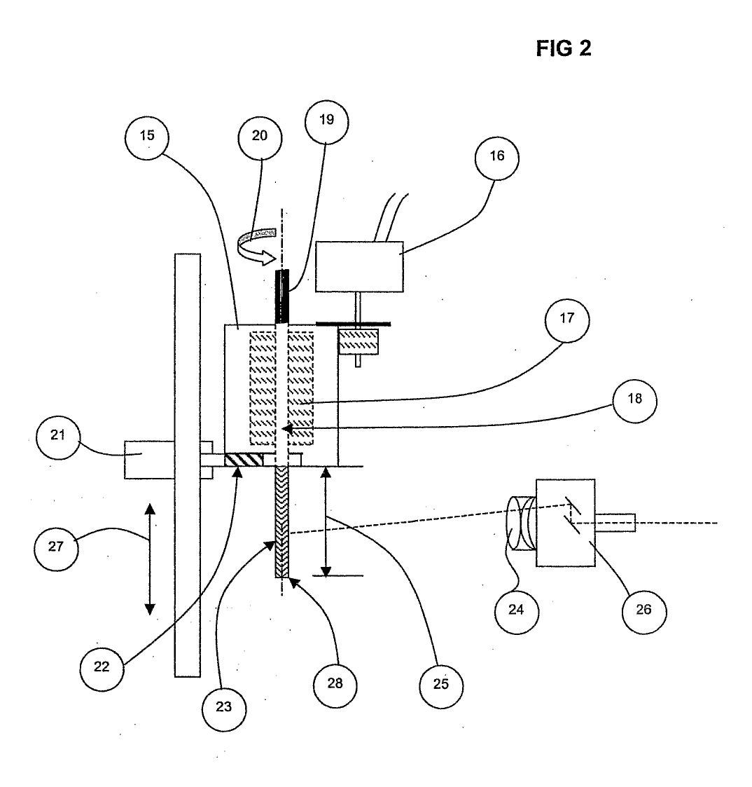

This example describes a station (15) for machining stent blanks (19), with a stent blank (19) placed at the centre (18) of the station and rotated (20) about its central axis with a movable stent fastener (17), which receives its motion from a linear or step motor (16). The machining station is illustrated in FIG. 2.

In accordance with the invention, a stent blank (19) is machined with a fibre-reinforced diode-pumped picosecond laser, from where the beam is conducted over the fibre to an optically corrected (24) scanner (26), allowing the entire stent length (25) to be machined as a finished stent (28) merely by rotating (20) the stent blank (19) about its own axis. Such a trajectory is very easy to control with a precision of ±μm. In accordance with the invention, it is no longer necessary to consider the vertical movement, since this is taken care of with a multiplied optically corrected planar scanner (24) and (26). When the stent (23) is finished, the vertical linear (21) grips the stent blank (19) and shifts (27) it (19) to the desired height (28), remaining, if desired, in support of the stent blank (19) on the axis (22) penetrating into the stent blank (19).

The stent blank (19) is moved only about its own central axis by about 0.5° per step pulse. With a 100 W net laser power, there will be about 36 pulses per second, the movement being regular enough for the stent blank not to shift its position during an overall machining moment of say, about 36 seconds.

With the entire process performed in a closed space without using any kind of cooling gases, no air currents will affect the piece or the precision during the cutting process. The picosecond laser used in the method of the invention will not either have any thermal effect on the stent blank in the process. Thus there will be no stresses in the stent material. This is why the stent blank does not have to be supported at its free end.

The issue above should be emphasised, because it should be understood why the prior art references describe support of the stent/stent blank during the work process.

The stent blank (19) and especially the machining area of the stent (23) have required even strong support in currently known laser applications, because, firstly, a rapid x-axis motion and a reciprocating y-motion have a substantial impact on the physical position of the stent. Secondly, precisely the thermal shock generated by the laser and the stress generated in the stent tend to modify the laser focus point substantially, thus resulting in cutting inaccuracy. The use of gas flows for reinforcing or cooling the laser beam, as disclosed in the prior art references, will have a substantial impact on the stability of the stent position. This naturally has a very detrimental effect on the laser operation, because the laser beam focus will not either be correct. The method described here consequently allows all these problems to be resolved.

The stent-manufacturing module shown in FIG. 2 is located in a sealed controlled space, e.g. a vacuum chamber. The pattern (1) and the production of the stent pattern produced by laser in the stent blank (19) do not generate heat, even though the temperature of the material plasma is typically about +1 million° K. this is due to the fact that the heat is totally bound to the atoms removed from the stent (23) by evaporation and subsequently removed from the chamber by vacuum ventilation, usually suction.

In front of an optically (24) corrected planar scanner (26), one preferably places either a) a negatively charged electric field, and then the volatile atoms will not pass in that direction, and/or b) a cassette comprising an automatically wound optic plastic film, which proceeds gradually as it is fouled. This is a solution to the problem of keeping the laser apparatus optics constantly clean, without restrictions to directing a laser beam to the stent area (25).

### Example 3

This example is a more detailed description of the transfers in FIG. 1, i.e. the operation of the setting unit (7), FIG. 3. The setting unit is in a controlled state, within a vacuum chamber (29), for instance, in which a stent cassette (30) is provided for vertically positioned stent blanks (31), which rotates about e.g. its own central axis, the stent blank (31) bearing e.g. against the bottom (40) of the stent cassette (30). Each time, the stent cassette (30) brings another stent blank (31) in advance to the transfer and setting area, where an engagement mechanism (32) is provided for gripping the stent by means of its jaws (39). The jaws (39) are placed in a motorised (33) central body (35), which is capable of shifting the stent blank totally dimensionally (34) in the plane (36) contacting the linear (37) bringing (38) the stent blank to the machining station.

### Example 4

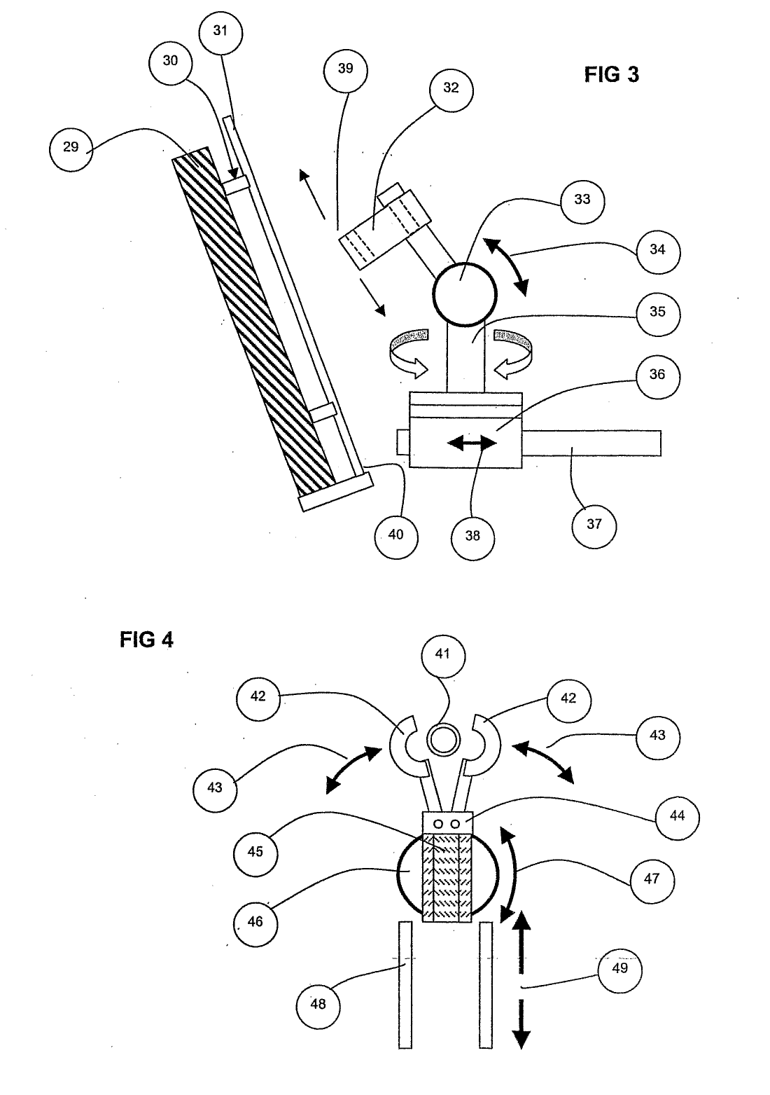

This example describes the operation of the setting unit viewed from above, FIG. 4. A circular stent blank (41) is preferably maintained slightly oblique in the stent cassette. In accordance with the invention, the stent setting unit and transfer device are preferably capable of performing any trajectories (43, 47 and 49) under completely three-dimensional parameters.

According to the exemplified operation principle, the jaws (42) engage the stent blank (41) by closing (43) towards each other, and then the body (46), which the jaw mechanism (42) has engaged, turns over e.g. (47) 180° about its own central axis (45) and moves by means of a linear conveyor (48) towards the stent machining station (49), from where it returns automatically to fetch the subsequent stent blank (41).

### Example 5

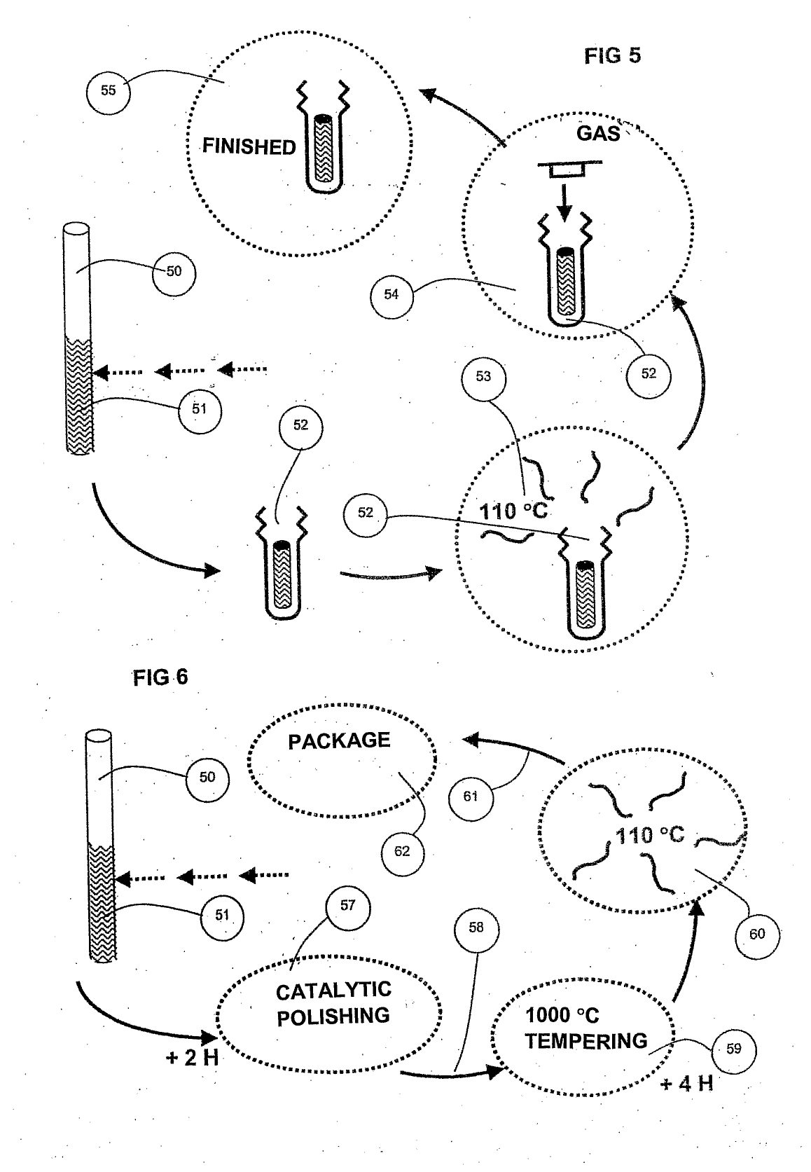

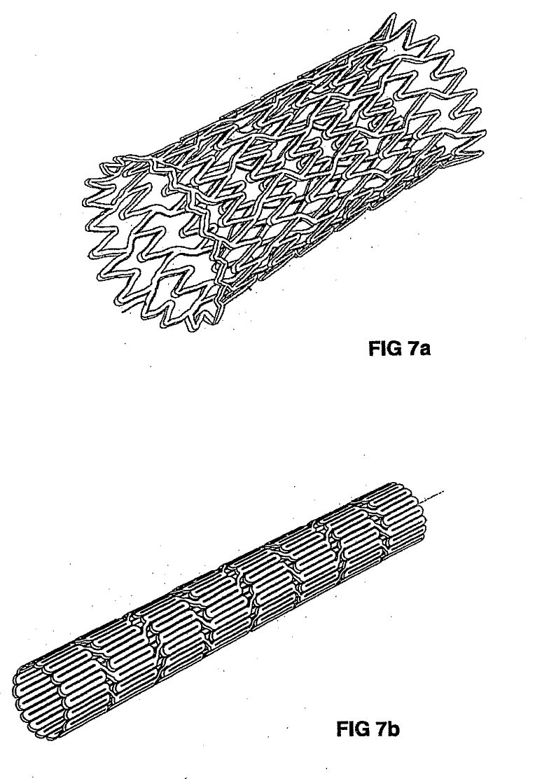

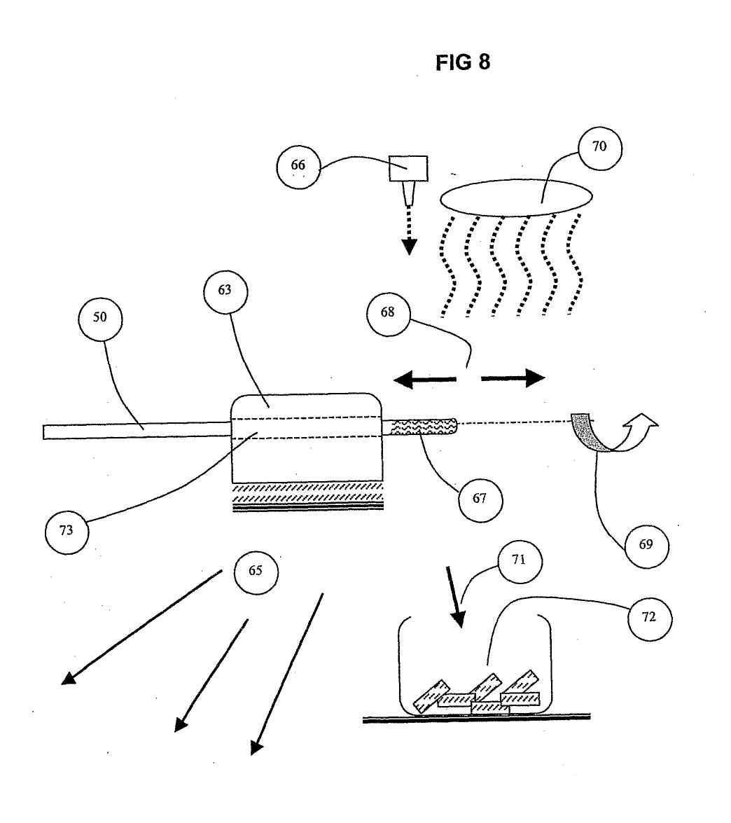

Example 5 depicts the work process of the method of the invention, FIG. 5. This method is substantially different both with respect to known processes for manufacturing stents and for the further processing of stents. The significant difference resides in the fact that the stent tube (50) is in “a preheated” form, i.e. it has its definitive softness. The stent tube is also pre-polished.

One of the benefits gained by the method is consequently that no further processing steps are required as in prior art methods.

When the laser of the new method has performed the engraving work process (51), which is based on material evaporation at a very high temperature (approximately +1 million° K (Kelvin)), it will not damage the stent in any way during the manufacturing process.

If the package made for the stent is also placed in a sealed work space, FIG. 1 (1), under gas atmosphere, the stent can be placed directly in the package (52) and (54) it can be packaged and (55) sealed. In this case, the entire stent cassette (2), with inserted (3) stent blanks, is sterilised as such, as are the inserted packages, which are located in their own cassette. The actual chamber (1) has then also been sterilised by a) UV light, b) gas and/or c) heat.

It is particularly easy to use UV light in sterilisation, because with a chamber FIG. 1 (1) made of stainless steel, the UV light will be reflected everywhere. A combination of UV light and protective gas will result in a 100% sterile space.

A second application comprises the automatic sterilisation illustrated in FIG. 5, in which the finished stent is placed in a package (52) and is moved, placed in a cassette, for instance, to an autoclave (53), which is filled with protective gas (54), the lid is closed and the product is finished (55).

### Example 6

Example 6 illustrates prior art steps for manufacturing a stent, FIG. 6. In such a method, a pattern is cut through the material wall of a hard stent tube (50). Burrs, i.e. irregular cutting traces have been produced in the preceding work process (51). The surface has become coarse and there will remain burnt metal fragments, flashes and sprays adhered, whose removal require the stents to be subjected to an electro-catalytic polishing process (57). Then the stents are transferred (58) to a tempering furnace (59), whose temperature is gradually raised to approximately +900-+1000° C. for the metal to resume its original softness, i.e. mouldability. Since the transfer between the work processes is performed manually and the stents are exposed to free ambient air, they require sterilisation e.g. in step (60), where a sealable package of glass or plastic has been placed.

### Example 7

Example 7 illustrates a prior art apparatus for manufacturing a stent, FIG. 8. Such a stent manufacturing machine (63) is typically equipped with two electric motors, e.g. a linear and a step motor, allowing the necessary high-precision work movements (68) and (69) to be carried out. These movements comprise a reciprocating path (68) and a rotary movement (69), which have been programmed in synchronisation such that the laser beam (66) incidence at the desired location is as accurate as possible.

The application of an optically corrected scanner (70) has been described above, and this is an improvement as such, since no reciprocating linear movement (68) is required, so that in theory, this should result in a more regular cutting trace, without, however, implying higher stent quality.

The use of an Nd:YAG laser has also been described above, causing the problems described above, with further processing performed in horizontal position (73) and manufacture in an open space (65). Also, the stents (67) are dropped (71) into a common recipient (72).

In prior art stent manufacturing methods, the stent apparatus (63) is constantly in horizontal position, as is the stent blank (64), FIG. 8. This means that the laser work processes (68), with a point beam requiring longitudinal reciprocating shifting (68) of the stent blank (64) and a scanner performing the work process over the entire length of the stent (67), are carried out horizontally. The stent will be most unstable with the work performed in horizontal position, since the work piece, the stent (57) tends to move downwards under the gravitational force (65) of the earth, and since a rotary movement (69) and a reciprocating movement (68) are performed, and since stresses are generated in the stent due to the work processes (60) and (70). This is why prior art methods have used different forms of support systems penetrating into the stent, since otherwise, it would be extremely difficult to carry out the laser cutting processes (66) and (70).

The subsequent work step comprises detaching the stent (67) from the stent blank (64), the stent dropping freely 871) into a box, where the stents (72) are mixed.

This is followed by the work steps illustrated in FIG. 6, which all require manual operations, since it is very difficult to automate work processes that have not been devised as such initially.

1. A method for preparing a stent, in which stent materials are machined by laser, characterised in that the stent blank is subjected to a work process, in which the desired pattern is cut quickly through the stent blank by evaporating the stent material with a diode-pumped fibre laser pulses having a repetition frequency above 1 MHz, wherein said laser is a picosecond or femtosecond laser.

2. A method as defined in claim 1, characterised in that the diode-pumped laser has a minimum power of 20 W, preferably a minimum power of 50 W and most advantageously a minimum power of 100 W.

3. A method as defined in claim 1, characterised in that the picosecond laser is a modularly reinforced and distributed fibre-reinforced picosecond laser.

4. A method as defined in claim 2, characterised in that the pulse adopted by the diode-pumped picosecond fibre laser has a repetition frequency preferably above 10 MHz and most advantageously above 40 MHz.

5. A method as defined in claim 1, characterised in that the stent blank has been made of metal or a metal compound.

6. A method as defined in claim 5, characterised in that the stent blank is preheated to a soft state before the pattern is cut through the stent blank by a diode-pumped fibre laser.

7. A method as defined in claim 1, characterised in that the stent blank is made of polymer, biopolymer or a ceramic material.

8. A method as defined in claim 1, characterised in that the method uses an optically corrected planar scanner, by means of which a laser beam is directed to a work piece, i.e. a stent blank.

9. A method as defined in claim 1, characterised in that the work process is performed with the work piece in vertical position.

10. A method as defined in claim 1, characterised in that the method uses an automated stent blank reserve.

11. A method as defined in claim 1, characterised in that all the work processes are automated and comprise all the necessary work processes including packaging.

12. A method as defined in claim 1, characterised in that the work space is a sealed vacuum chamber, where the process may take place under gas atmosphere, vacuum, pressure, or with a combined or joint use of these.

13. A method as defined in claim 1, characterised in that the stent blank can be machined over its entire length and cut to its proper length only after this.

14. A stent, characterised in that the stent has a well-defined cutting line and that it is manufactured according to method claim 1.

15. A method as defined in claim 2, characterised in that the picosecond laser is a modularly reinforced and distributed fibre-reinforced picosecond laser.

16. A method as defined in claim 3, characterised in that the pulse adopted by the diode-pumped picosecond fibre laser has a repetition frequency preferably above 10 MHz and most advantageously above 40 MHz.

17. A stent, characterised in that the stent has a well-defined cutting line and that it is manufactured according to method claim 2.

18. A stent, characterised in that the stent has a well-defined cutting line and that it is manufactured according to method claim 3.

19. A stent, characterised in that the stent has a well-defined cutting line and that it is manufactured according to method claim 4.

20. A stent, characterised in that the stent has a well-defined cutting line and that it is manufactured according to method claim 5.
